(19) United States
(12) Patent Application Publication
(10) Pub. No.: US 2009/0299087 A1
(43) Pub. Date: Dec. 3, 2009

(54) Process for manufacturing unsaturated mononitriles to improve on-stream time and reduce fouling

(75) Inventors: Valerie S. Monical, Houston, TX (US); Michael Joseph Heniff, JR., Pearland, TX (US); Scott Gordon Moffatt, Pearland, TX (US); Richard D. Murphy, League City, TX (US); Gregory Ward, Katy, TX (US)

Correspondence Address: MORGAN LEWIS & BOCKIUS LLP, 1111 PENNSYLVANIA AVENUE NW, WASHINGTON, DC, 20004, US

(73) Assignee: Solutia, Inc., St. Louis, MO (US)

(21) Appl. No.: 12/153,936

(22) Filed: May 28, 2008

(51) Int. Cl. C07C 253/34; C07C 253/26

(52) U.S. Cl. 558/320; 558/466

(57) ABSTRACT

The process for manufacturing unsaturated mononitriles, such as acrylonitrile and methacrylonitrile, has been modified to add a partially condensed quench effluent stripper column to remove high boiling organic compounds from the reactor effluent prior to introduction into the extractive distillation recovery column. The high boiling organic compounds are preferably removed after the ammonia in the reactor effluent has been neutralized and the neutralization products have been removed. The targeted high boiling organic compounds are associated with fouling in the recovery section of the plant.

### BACKGROUND OF THE INVENTION

1. Field of the Invention

The present invention generally relates to the addition of a partially condensed quench effluent stripper column to a process for manufacturing unsaturated mononitrile, such as acrylonitrile or methacrylonitrile, in order to remove high boiling organic compounds.

2. Description of Related Art

In commercial processes for preparation of acrylonitrile from propylene, ammonia, and oxygen (air), the reactor effluent contains, in addition to the desired acrylonitrile (AN) product, considerable amounts of by-product hydrogen cyanide (HCN), acetonitrile, and other impurities include high boiling organic compounds. These high boiling organic compounds (also referred to as “heavies”) have been shown to include fumaronitrile, maleonitrile, acrylic acid, and derivatives of acrolein. The exact composition of the effluent and the by-products and impurities it contains may vary considerably depending on the ammoxidation reaction conditions and catalyst.

Processes for treating reactor effluents of the type described to separate and recover acrylonitrile product and desired by-products such as hydrogen cyanide and acetonitrile are known. For example, see U.S. Pat. Nos. 3,399,120; 3,433,822; 3,936,360; 4,059,492; 4,166,008; and 4,404,064, the disclosures of which are incorporated herein by reference. Typically, these processes include introducing the reactor effluent into a quench chamber where it is contacted with water (usually containing sulfuric acid to neutralize excess ammonia from the reaction) to cool the effluent and remove some contaminates such as high boiling impurities produced in the reactor. Cooled effluent gases from the quench flow to an absorber column where they are contacted with water. The liquid stream from the bottom of the absorber column contains most of the nitrites produced in the reaction and impurities and is sent to an extractive distillation column. The major portion of the acrylonitrile from the extractive distillation column is obtained in the overhead (distillate) from the column while water and impurities constitute the bottom stream from the column. In accordance with practices of the art, the bottom stream is frequently fed to a secondary distillation or stripper column to separate acetonitrile and water in an overhead stream while the secondary column bottoms containing water and various impurities are recycled to the absorber column.

Acrylonitrile manufacturing plants may include either a cold quenching system or a hot quenching system. When a cold quenching system is used, a significant amount of heavies (approximately 80-90%) are purged at the quench column and routed to a waste treatment or disposal system which often includes a deepwell. In the hot quench system, many of the heavies (approximately 80-90%) remain in the vapor phase and are carried-over from the quench column to the absorber and recovery column. Once in recovery, some heavies are poorly purged resulting in their accumulation and resultant fouling.

Unfortunately, under operating conditions, many components in an acrylonitrile plant can polymerize in the recovery and purification sections to form solid deposits which interfere with operation of equipment, contribute to an undesirable net production loss and reduction in production rates, and with time lead to costly shutdowns. Fouling in the recovery section of acrylonitrile manufacturing plants is a costly problem resulting in manufacturing downtime and maintenance costs.

### BRIEF SUMMARY OF THE INVENTION

The process for manufacturing unsaturated mononitriles, such as acrylonitrile or methacrylonitrile, has been modified to add a partially condensed quench effluent stripper column to remove high boiling organic compounds from the reactor effluent prior to introduction into the extractive distillation recovery column. The high boiling organic compounds are preferably removed after the ammonia in the reactor effluent has been neutralized and the neutralization products have been removed by partial condensation of the quench vapor effluent. The partial condensation may be accomplished by either direct contact cooling or indirect cooling through a heat exchanger. The targeted high boiling organic compounds are associated with fouling in the recovery section of the plant.

In a preferred embodiment, the partially condensed quench effluent stripper column is added to receive a portion of the partially condensed quench effluent that would normally be fed to a standard absorber traditionally used in an acrylonitrile manufacturing process. The addition of a partially condensed quench effluent stripper column to the acrylonitrile manufacturing plant yields a significant reduction in process fouling thereby increasing the run time of the recovery and stripper columns.

Although the present invention seeks to provide several benefits to the acrylonitrile, or methacrylonitrile, manufacturing process, other benefits that are not expressly mentioned herein are readily ascertainable to those skilled in the art. The present invention prevents high boiling compounds from concentrating in the large water streams in the plant. In one embodiment, the high boiling organic compounds can be easily separated from the process stream using a partially condensed quench effluent stripper column with a relatively small diameter and a few stages. The entire acrylonitrile manufacturing plant can be operated with or without the added partially condensed quench effluent stripper column so that cleaning the partially condensed quench effluent stripper column does not require a complete shutdown of the facility. The acrylonitrile product quality is improved by increased purging of high boiling organic compounds. Generally, the operating costs for the partially condensed quench effluent stripper column are relatively low. Because high boiling organic compounds are removed, the recovery section of the manufacturing plant is subjected to reduced amounts of fouling that further reduces the downtime for the rest of the facility.

### BRIEF DESCRIPTION OF THE DRAWINGS

The features and advantage of the present invention will become apparent from the following detailed description of a preferred embodiment thereof, taken in conjunction with the accompanying drawing, in which:

FIG. 1 depicts a general block flow diagram of one embodiment of the present invention wherein a partially condensed quench effluent stripper column is added to an acrylonitrile manufacturing process.

### DETAILED DESCRIPTION OF THE INVENTION

The present invention includes a method for removing high boiling organic compounds from any known reactor product effluent resulting from the catalytic oxidation of an olefin and ammonia in the presence of an oxygen source. One such well known commercial process includes the production of acrylonitrile by ammoxidation of propylene with ammonia in the presence of an oxidation catalyst. It should be understood that any mention of acrylonitrile in this specification should be construed to also include other acceptable unsaturated mononitriles, such as methacrylonitrile. The product effluent of such reaction normally contains, in addition to acrylonitrile, by-products hydrogen cyanide, acetonitrile, acrolein, acrylic acid, and high boiling organic compounds. For purposes of this invention, the phrase “high boiling organic compounds” is defined as an organic compound having a boiling point above the boiling point of acrylonitrile. More specifically, “high boiling organic compounds” are organic compounds having a boiling point of 78° C. or higher. “High boiling organic compounds” may be categorized as including “light high boiling organic compounds” and “heavy high boiling organic compounds”. “Light high boiling organic compounds” include organic compounds having a boiling point higher than the boiling point of acrylonitrile (normal boiling point (nbp) of 78° C.) and up to and including the boiling point of fumaronitrile (nbp of 186° C.). “Heavy high boiling organic compounds” include organic compounds having a boiling point higher than the boiling point of fumaronitrile (nbp of 186° C.). “Heavy high boiling organic compounds” include acrylamide and succinonitrile. Patents claiming specific catalysts and processes for their use in the manufacturing of acrylonitrile and methacrylonitrile by the ammoxidation of propylene and isobutylene, respectively, include U.S. Pat. Nos. 2,481,826; 2,904,580; 3,044,966; 3,050,546; 3,197,419; 3,198,750; 3,200,084; 3,230,246; 3,248,340; and 3,352,764, all of which are incorporated herein by reference. It is understood that the present invention is not dependent upon any specific ammoxidation fluid bed catalyst. Suitable catalysts which are more selective for the ammoxidation of propylene and isobutylene can be prepared from bismuth, cobalt, iron, nickel, tin salts, and molybdic, molybdic phosphoric, and molybdic silicic acids. Other components, such as tungsten, copper, tellurium, and arsenic oxides, have been incorporated to increase low temperature activity and productivity.

Effluent from the ammoxidation reactor is cooled in a quench tower. In one embodiment, reactor effluent may be cooled with an acidified water stream by counter-current contact. Gases from the quench tower are preferably transferred into the bottom of an absorber where acrylonitrile, hydrogen cyanide, acetonitrile, and other soluble gases are absorbed in water to provide an aqueous solution. The non-absorbed gases are vented.

A stream from the absorber, known as the rich water stream, is transferred into a recovery column where it is extractively distilled. The recovery column may be any suitable contacting means in which liquid and vapor are counter-currently contacted in a multiplicity of communicating zones or stages. The overhead vapors from the recovery column contain mainly acrylonitrile (AN) and hydrogen cyanide (HCN) with some water and impurities that are fed to a heads column where HCN is removed as a product overhead. The bottom water and AN stream is sent to a drying column with a drying column decanter to remove the water and recycle it back to the recovery column feed. The drying column bottoms are then sent to a product column where heavy organic compounds are removed and the AN is recovered as a virtually pure product. However, it should be understood that other methods and techniques may be used to recover the AN from the overhead vapors of the recovery column and that the present invention is not intended to be limited to the separation technique previously described. One of ordinary skill in the art can ascertain which types of separation methods and techniques would be suitable to separated the AN in the overhead vapors of the recovery column.

A partially condensed quench effluent including a substantial amount of the high boiling organic compounds is removed from the aqueous solution and sent to a partially condensed quench effluent stripper column to remove high boiling organic compounds from the process stream. Preferably, a distillation column or a stripper is introduced to receive partially condensed quench effluent from a standard absorber that has been modified to include a partial condensing section operating between 80-155° F. The stripper may be operated with live steam for direct stripping and/or with a reboiler. Optionally, a rectification section could be added to the stripper. In a preferred embodiment, the stripper may include as few as 10 and up to 50 trays. The partially condensed quench effluent stripper column receives the partially condensed quench effluent and separates AN and HCN from the high boiling organic compounds. These high boiling organic compounds generally include fumaronitrile, maleonitrile acrylic acid, and derivatives of acrolein. The distillate from the partially condensed quench effluent stripper column includes recovered hydrogen cyanide (HCN) and acrylonitrile (AN) that is preferably returned to the manufacturing process. In a preferred embodiment, the distillate may be fed to the quencher, the absorber, the feed of the heads column, and/or the feed of the recovery column. It is understood that one of skill in the art would be able to ascertain other process steps within the manufacturing process that the distillate may be returned. This process for removing the high boiling organic compounds reduces fouling and improves on-stream time of the recovery column.

The invention is described by reference to the drawing. However, those skilled in the art will appreciate that many variations of the specific separation process depicted are known and that the essence of this invention—the addition of a partially condensed quench effluent stripper column—can be beneficially applied to any such variation.

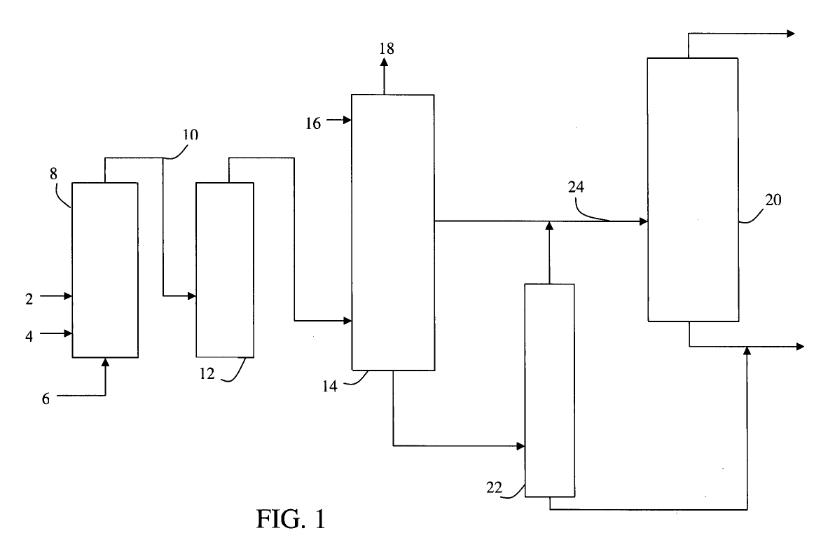

FIG. 1 depicts a basic block flow diagram of one embodiment of the present invention as applied to an acrylonitrile manufacturing process. As in conventional practice, propylene 2 and ammonia 4 are reacted with air 6 (or oxygen) over a fluidized catalyst in a reactor 8 to make acrylonitrile (AN), hydrogen cyanide (HCN) and other impurities. The ammoxidation reactor effluent 10 is directed to a quencher 12 to neutralize the unreacted ammonia and remove any carried over catalyst from the system. The quencher 12 may be a cold quench design including a mechanism, such as a heat exchanger, to remove heat from the reactor effluent. However, the present invention is directed primarily to hot quench designs where little or no heat is removed during the quench process. In the cold quench, a significant amount of light high boiling organic compounds are purged at the quench column and routed to a process to recover or treat the waste, which may include a deepwell. In the hot quench, most of the light high boiling organic compounds are carried-over from the quench column to the recovery stage. Since the hot quench will include more light high boiling organic compounds than the cold quench, the present invention is more advantageous in an acrylonitrile manufacturing process that uses a hot quench design. However, the present invention may provide some benefits in removing light high boiling organic compounds in an acrylonitrile manufacturing process that uses cold quench. The product stream from the quencher 12 is directed to an absorber 14 where water 16 is added to condense/absorb the AN and HCN in water providing an aqueous solution, rejecting the non-condensible reaction feeds (i.e. propylene, propane, and nitrogen) in an off-gas stream 18. The condensed AN and HCN in water are fed to an extractive distillation recovery column 20 to remove the impurities. It is noted that the designs of extractive distillation recovery columns are varied and frequently employ heat recovery devices and use recycle streams from point to point in the column or from other process units to optimize separation efficiency and/or economy. The design of the extractive distillation recovery column and of the previously referenced quench and absorber columns are not critical to this invention and any commercially viable design can be utilized. In general, in extractive distillation columns, water is introduced (usually located above the feed point of the bottoms stream from the absorber) to effect extractive distillation in the column which will normally contain 50-100 or more separation stages.

In accordance with a preferred embodiment of the present invention, a partially condensed quench effluent containing a substantial amount of the high boiling organic compounds but only 1-15% of the product AN is removed as an aqueous solution in the absorber 14 and sent to the partially condensed quench effluent stripper column 22 to remove high boiling organic compounds with an aqueous bottoms phase, and overheads including both an organic phase and an aqueous phase containing recovered HCN and AN are fed to the recovery column 24. However, it is appreciated that the organic phase and aqueous phase in the overheads may be separated and further processed. This partially condensed quench effluent preferably contains between 2-10% by weight AN and 0.25-1% by weight HCN, and 1-3% high boiling organic compounds. Additionally, this partially condensed quench effluent will be high in water content, preferably higher than 90%. The high boiling organic compounds in the partially condensed quench effluent comprise 80-95% of the high boiling organic compounds in the aqueous solution in the absorber 14. The partially condensed quench effluent stripper column 22 generally serves to split the HCN and AN as overhead that is returned to the feed of the recovery column 24 from the high boiling organic compounds (including succinonitile, AMS and acrolein derivatives). The distillate from the partially condensed quench effluent stripper column generally contains a very low level of high boiling organic compounds, typically less than 2% of the high boiling organic compounds fed to the partially condensed quench effluent stripper column, preferably less than 0.5% of the high boiling organic compounds fed to the partially condensed quench effluent stripper column.

In one embodiment of the present invention, ammonia from the reactor 8 is neutralized by sulfuric acid with only adiabatic cooling. The vapor stream resulting from this process is cooled either in a partial condenser or in a trayed column section with a cooled pumparound. The aqueous solution product from the cooling operation is sent to the partially condensed quench effluent stripper column 22 to separate the desirable products (AN and HCN) from a significant fraction of the high boiling organic compounds (including acrylic acid, derivatives of acrolein, fumaronitrile, maleonitrile, etc.). The separation of acetonitrile can be manipulated to be partially removed with the high boiling organic compounds or recovered with the products. The recovered products are returned to the absorber or recovery column in the traditional manufacturing process.

In another preferred embodiment, a portion of the condensed liquid from an indirect contact cooler as describer in U.S. Pat. No. 4,234,510 may be feed to the partially condensed quench effluent stripper column 22. The contents of U.S. Pat. No. 4,234,510 are expressly incorporated herein by reference. As described in U.S. Pat. No. 4,234,510, the reactor effluent is subjected to a cold quench cooled by direct contact cooling and partial condensation is accomplished with a heat exchanger. It is submitted that one of skill in the art would appreciate that the present invention may be used to remove high boiling organic compounds in system having hot quench/direct contact partial condensation, cold quench/indirect heat transfer for partial condensation, hot quench/indirect partial condensation, or cold quench/direct contact partial condensation. Preferably, the partially condensed quench effluent may be formed from indirect heat exchange or direct contact cooling.

Those skilled in the art will appreciate that all columns will be provided with necessary heat to effect their intended functions and that, for purposes of economy, much of such heat will be obtained from recycle streams used to supply processing liquid to the columns or to provide improved concentrations/separation of various components. Such recycle and heat recovery techniques are conventional practice and, for simplicity, are not shown in the drawings or discussed in detail herein.

The invention is further illustrated by the following example:

### EXAMPLE

Partially condensed quench effluent from two absorbers are used. The first partially condensed quench effluent includes the following components provided at the following flow-rates:

TABLE 1

| | Lb/h | % (Wt) |
|---|---|---|
| HCN | 339 | 0.44 |
| Acrylonitrile | 2443 | 3.15 |
| Acetonitrile | 76 | 0.10 |
| Water | 73,548 | 94.92 |
| Light High boiling | 376 | 0.49 |
| organic compounds | | |
| Heavy high boiling | 355 | 0.46 |
| organic compounds | | |
| Succinonitrile | 7 | 0.01 |
| Acrolein Derivatives | 153 | 0.20 |
| AMS | 190 | 0.25 |

The second partially condensed quench effluent includes the following components provided at the following flow rates:

TABLE 2

| | Lb/h | % (Wt) |
|---|---|---|
| HCN | 334 | 0.44 |
| Acrylonitrile | 2181 | 2.86 |
| Acetonitrile | 74 | 0.10 |
| Water | 72,596 | 95.21 |
| Light high boiling organic | 366 | 0.48 |
| compounds | | |
| Heavy high boiling | 352 | 0.46 |
| organic compounds | | |
| Succinonitrile | 7 | 0.01 |
| Acrolein Derivatives | 153 | 0.20 |
| AMS | 186 | 0.24 |

The following parameters apply to each of the partially condensed quench effluent streams:

| | |
|---|---|
| Maximum flow: 100,000 lb/hr | Temperature: 144° F. |
| Pressure: 180.0 psia | Density: 60.5 lb/ft³ |
| Viscosity: 0.46 cP | pH: 4.0 to 5.0 |

The first and second partially condensed quench effluent streams are combined to provide a single feed to the partially condensed quench effluent stripper column having the following components and flow-rates:

TABLE 3

| | Lb/h | % (Wt) |
|---|---|---|
| HCN | 673 | 0.44 |
| Acrylonitrile | 4624 | 3.01 |
| Acetonitrile | 150 | 0.10 |
| Water | 146,144 | 95.06 |
| Light high boiling organic | 742 | 0.48 |
| compounds | | |
| Heavy high boiling | 707 | 0.46 |
| organic compounds | | |
| Succinonitrile | 14.0 | 0.01 |
| Acrolein Derivatives | 306 | 0.20 |
| AMS | 376 | 0.24 |

The following parameters apply to the feed for the quench separation column:

| | |
|---|---|
| Maximum flow: 200,000 lb/hr | Temperature: 144° F. |
| Pressure: 180.0 psia | Density: 60.5 lb/ft³ |
| Viscosity: 0.46 cP | pH: 4.0 to 5.0 |

The partially condensed quench effluent stripper column is a 33-tray steam distillation column. The feed is introduced into the partially condensed quench effluent stripper column at tray 33. The partially condensed quench effluent stripper column is operated such that the control tray location is tray 10 having a temperature of 189° F. This location provides a stable control point that reduces large stream flow swings. The bottoms from the partially condensed quench effluent stripper column containing the high boiling organic compounds is sent to a waste water deep well. The components and the flow-rate of the bottoms of the partially condensed quench effluent stripper column are:

TABLE 4

| | Lb/h | % (Wt) |
|---|---|---|
| HCN | 20 | 0.01 |
| Acrylonitrile | 102 | 0.07 |
| Acetonitrile | 128 | 0.09 |
| Water | 145,184 | 98.39 |
| Light high boiling organic | 737 | 0.50 |
| compounds | | |
| Heavy high boiling organic | 707 | 0.48 |
| compounds | | |
| Succinonitrile | 14.0 | 0.01 |
| Acrolein Deriv. | 286 | 0.19 |
| AMS | 376 | 0.25 |

The following parameters apply to the bottoms of the partially condensed quench effluent stripper column:

| | |
|---|---|
| Maximum flow: 207,000 lb/hr | Temperature: 228° F. |
| Density: 58.6 lb/ft³ | Viscosity: 0.24 cP |

The overhead from the partially condensed quench effluent stripper column is sent to a scrubber where off-gases are further sent to a process flare heater. It is appreciated that one of skill in the art would be able to select an appropriate scrubber. The resulting distillate from the partially condensed quench effluent stripper column has the following components and flow-rates:

TABLE 5

| | Lb/h | % (Wt) |
|---|---|---|
| HCN | 653 | 8.5 |
| Acrylonitrile | 4522 | 58.87 |
| Acetonitrile | 22 | 0.36 |
| Water | 2459 | 32.02 |
| Light high boiling organic | 5 | 0.08 |
| compounds | | |
| Heavy high boiling organic | 0 | 0 |
| compounds | | |
| Succinonitrile | 0 | 0 |
| Acrolein Deriv. | 20 | 0.26 |
| AMS | 0 | 0 |

The following parameters apply to the distillate of the partially condensed quench effluent stripper column:

| | |
|---|---|
| Temperature: 168° F. | Pressure: 17 psia |
| Density: 0.096 lb/ft³ | Viscosity: 0.01 cP |

The distillate from the partially condensed quench effluent stripper column is combined with the rich water streams from the absorbers and directed to the recovery feed of the Extraction Distillation Recovery Column where the AN and HCN are removed as overhead vapor for further separation to recover a purified amount of AN.

Although the present invention has been disclosed in terms of a preferred embodiment, it will be understood that numerous additional modifications and variations could be made thereto without departing from the scope of the invention as defined by the following claims:

We claim:

1. In a process for purifying an unsaturated mononitrile in which an ammoxidation reactor effluent containing an unsaturated mononitrile and high boiling organic compounds is quenched and condensed to provide an aqueous solution: the improvement being that a portion of the aqueous solution containing a substantial amount of the high boiling organic compounds is removed as partially condensed quench effluent and directed to a partially condensed quench effluent stripper column to remove high boiling organic compounds from said process.

2. The process of claim 1, wherein said high boiling organic compounds include compounds having a boiling point of 78° C. or higher.

3. The process of claim 1, wherein said unsaturated mononitrile is acrylonitrile.

4. The process of claim 3, wherein said partially condensed stream includes 2-10 wt. % acrylonitrile, 0.25-1 wt. % hydrogen cyanide, 1-3 wt. % high boiling organic compounds, and the remainder being water to total 100 wt %.

5. The process of claim 3, wherein said reactor effluent is quenched using a hot quenching technique.

6. The process of claim 5, wherein said partially condensed quench effluent contains 80-95% of the high boiling organic compounds from the aqueous solution.

7. The process of claim 3, wherein said partially condensed quench effluent stripper column provides a distillate that contains less than 2 wt. % high boiling organic compounds fed to the partially condensed quench effluent stripper column.

8. The process of claim 7, wherein said partially condensed quench effluent stripper column provides a distillate that contains less than 0.5 wt. % high boiling organic compounds fed to the partially condensed quench effluent stripper column.

9. The process of claim 1, further comprises an absorber including a partially condensed section operating at 80-155° F. providing the partially condensed quench effluent.

10. A process for manufacturing an unsaturated mononitrile which process comprises:
a) reacting at least one olefin with ammonia and a source of oxygen in the presence of a catalyst in a reactor to produce a reactor effluent containing the corresponding unsaturated mononitrile and high boiling organic compounds;
b) transferring the reactor effluent to a quencher to cool the effluent resulting in a quench effluent, and thereafter the quench effluent is contacted with an aqueous stream producing an aqueous solution; and
c) transferring a partially condensed quench effluent containing a substantial amount of the high boiling organic compounds from the aqueous solution to a partially condensed quench effluent stripper column to remove high boiling organic compounds from the process.

11. The process of claim 10, wherein said high boiling organic compounds include compounds having a boiling point of 78° C. or higher.

12. The process of claim 10, wherein said unsaturated mononitrile is acrylonitrile.

13. The process of claim 12, wherein said partially condensed quench effluent includes 2-10 wt. % acrylonitrile, 0.25-1 wt. % hydrogen cyanide, 1-3 wt. % high boiling organic compounds, and the remainder being water to total 100 wt %.

14. The process of claim 12, wherein said reactor effluent is quenched using a hot quenching technique.

15. The process of claim 14, wherein said partially condensed quench effluent contains 80-95% of the high boiling organic compounds from the aqueous solution.

16. The process of claim 12, wherein said partially condensed quench effluent stripper column provides a distillate that contains less than 2 wt. % high boiling organic compounds fed to the partially condensed quench effluent stripper column.

17. The process of claim 16, wherein said partially condensed quench effluent stripper column provides a distillate that contains less than 0.5 wt. % high boiling organic compounds fed to the partially condensed quench effluent stripper column.

18. The process of claim 10, further comprising an absorber including a partially condensed section operating at 80-155° F. to provide the partially condensed quench effluent.

19. The process of claim 10, further comprising
d) transferring distillate from the partially condensed quench effluent stripper column to a recovery column.
